(19) United States
(12) Patent Application Publication
(10) Pub. No.: US 2010/0292538 A1
(43) Pub. Date: Nov. 18, 2010

(54) ENDOSCOPE

(76) Inventors: Hidetoshi HIRATA, Ashigarakami-gun (JP); Tsuyoshi Ashida, Ashigarakami-gun (JP); Takayuki Nakamura, Ashigarakami-gun (JP); Osamu Kuroda, Ashigarakami-gun (JP); Yasunori Ohta, Ashigarakami-gun (JP); Kazuyoshi Hara, Ashigarakami-gun (JP); Kazumi Koike, Saitama-shi (JP); Takuji Tada, Ashigarakami-gun (JP); Shinichi Yamakawa, Ashigarakami-gun (JP)

Correspondence Address: BIRCH STEWART KOLASCH & BIRCH, PO BOX 747, FALLS CHURCH, VA, 22040-0747, US

(21) Appl. No.: 12/777,571

(22) Filed: May 11, 2010

(30) Foreign Application Priority Data
May 12, 2009 (JP) P2009-115559

(51) Int. Cl. A61B 1/00

(52) U.S. Cl. 600/129

(57) ABSTRACT

A front end rigid portion constitutes a front end of an endoscope insertion unit in an endoscope. The front end rigid portion is provided with an illumination optical system irradiating an observation target area with illumination light and an imaging optical system having an imaging element imaging the observation target area. The endoscope includes a heat emission member which is connected to the front end rigid portion and extends in the longitudinal direction of the endoscope insertion unit. The heat emission member is formed by mixing fiber-piece-shaped fillers with a resin material.

### CROSS-REFERENCE TO RELATED APPLICATIONS

This application claims the benefit of Japanese Patent Application No. 2009-115559, filed on May 12, 2009, the entire contents of which are hereby incorporated by reference, the same as if set forth at length; the entire of which are incorporated herein by reference.

### BACKGROUND OF THE INVENTION

1. Technical Field

The present invention relates to an endoscope.

2. Description of Related Art

An endoscope includes a thin and long endoscope insertion unit which is inserted into a body cavity, and an endoscope front end as a front end of the endoscope insertion unit is provided with an illumination optical system illuminating an observation target area and an imaging optical system including an imaging element imaging the observation target area. The illumination optical system includes a light guide which is disposed along the inner wall of the endoscope insertion unit and is formed by a bundle of optical fibers. The base end of the light guide is connected to a light source device, light emitted from the light source device is guided to the endoscope front end, and the illumination light is emitted from the endoscope front end. In addition, the imaging optical system forms an observation image of the observation target area by using an object lens of the endoscope front end and the imaging element disposed at an image forming position of the object lens.

In the endoscope, when the imaging operation is performed by increasing the light amount of the illumination optical system, noise of the captured image can be reduced. In addition, since the aperture diameter of the imaging optical system can be decreased, that is, the F number can be increased, it is possible to obtain a high-quality image by adjusting a focus from a far position to a near position. For this reason, it is desirable to increase the luminance of the observation light source. Further, in recent years, there has been great demand for a further decrease in the thickness of the endoscope front end in order to realize insertion without any inconvenience. Additionally, there has been great demand for an increase in the number of pixels of the imaging element in order to perform more detailed observation.

However, an increase in the luminance of the observation light source and an increase in the number of pixels of the imaging element increase the heating amount of the endoscope, and a decrease in the thickness of the endoscope front end for improving the insertion of the endoscope deteriorates the heat emission property. For this reason, there is a concern in that an electronic device deteriorates or a signal noise increases. Due to these circumstances, various technologies for cooling the endoscope front end have been examined.

For example, JP-A-2008-029597 discloses a configuration in which a heat emission member is provided inside an endoscope front end so as to diffuse the heating generated from an illumination optical system or an imaging element. Further, JP-A-2006-198232, JP-A-2007-195824 and JP-A-2004-249099 disclose an example in which a carbon fiber is used for the constituent members of the endoscope.

### SUMMARY

An object of the invention is to provide an endoscope capable of realizing an increase in luminance of an observation light source and an increase in the number of pixels of an imaging element and thinning an endoscope front end by cooling a heat emission area of an endoscope front end.

The invention is shown in detailed below.

An endoscope includes an endoscope insertion unit and a heat emission member. The endoscope insertion unit includes a front end rigid portion constituting a front end of the endoscope insertion unit. The front end rigid portion is provided with (i) an illumination optical system irradiating an observation target area with illumination light and (ii) an imaging optical system having an imaging element imaging the observation target area. The heat emission member is connected to the front end rigid portion, and extends in the longitudinal direction of the endoscope insertion unit. The heat emission member is formed by mixing fiber-piece-shaped fillers with a resin material.

According to the endoscope of the invention, it is possible to diffuse the heating generated from the illumination optical system or the imaging optical system in the longitudinal direction of the endoscope insertion unit through the heat emission member, and thus to suppress an increase in the temperature of the endoscope front end. Accordingly, it is possible to prevent an increase in the noise signal of the imaging element or a deterioration in the performance, and thus to obtain a high-quality observation image. In addition, even when the endoscope front end unit is further thinned, it is possible to sufficiently reduce a thermal resistance in the longitudinal direction of the endoscope insertion unit.

### BRIEF DESCRIPTION OF THE DRAWINGS

FIG. 1 is an entire configuration diagram of an endoscope according to an embodiment of the invention.

FIG. 2 is a schematic external perspective view of a front end of an endoscope insertion unit shown in FIG. 1.

FIG. 3 is a schematic sectional configuration diagram taken along the line A-A of FIG. 2.

### DETAILED DESCRIPTION OF EMBODIMENTS OF THE INVENTION

Hereinafter, embodiments of the invention will be described in detail with reference to the accompanying drawings.

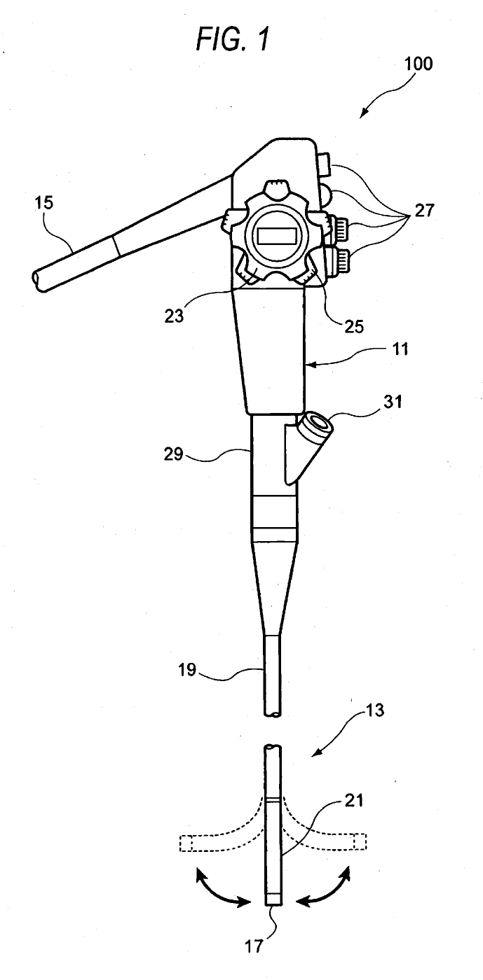

FIG. 1 is an entire configuration diagram of an endoscope according to an embodiment of the invention.

An endoscope 100 includes a body operation unit 11 and an endoscope insertion unit 13 which is connected to the body operation unit 11 and is inserted into a body cavity. A universal cord 15 is connected to the body operation unit 11, and a light guide (LG) connector (not shown) is provided in the front end of the universal cord 15. The LG connector is attachably/detachably connected to a light source device (not shown), thereby transmitting illumination light to an illumination optical system of a front end (hereinafter, referred to as an endoscope front end) 17 as a front end of the endoscope insertion unit 13. In addition, a video connector is connected to the LG connector, and the video connector is attachably/detachably connected to a processor for performing an image process and the like.

The endoscope insertion unit 13 includes a soft portion 19, a bending portion 21, and a front end 17 which are sequentially provided from the body operation unit 11. The bending portion 21 is remotely operated to be bent by the rotation of angle knobs 23 and 25 of the body operation unit 11. Accordingly, it is possible to direct the front end 17 to a desired direction.

The body operation unit 11 is provided with various buttons 27 such as an air sending button, a water sending button, a suction button, and a shutter button in addition to the above-described angle knobs 23 and 25. In addition, an extension portion 29 extending to the endoscope insertion unit 13 includes a clamp insertion portion 31. The clamp insertion portion 31 is used to draw out an inserted treatment tool such as a clamp from a clamp opening described below and provided in the front end 17 of the endoscope insertion unit 13.

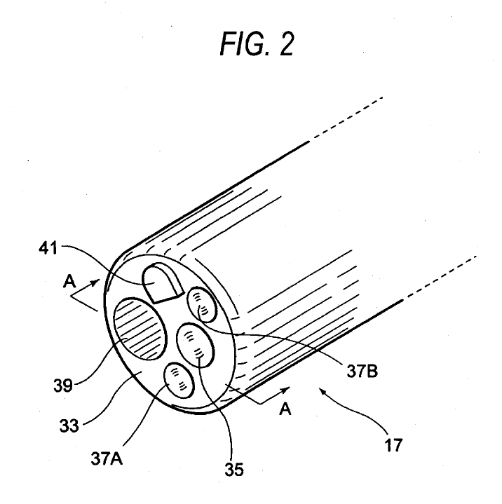

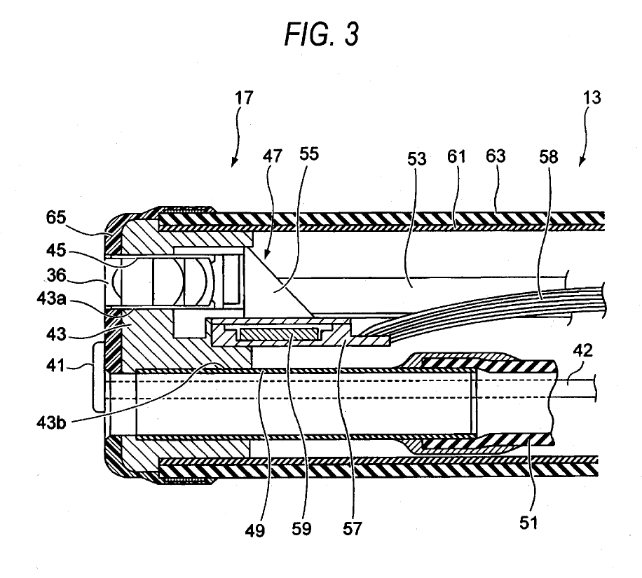

FIG. 2 is a schematic external perspective view of the front end of the endoscope insertion unit, and FIG. 3 is a schematic sectional configuration diagram taken along the line A-A of FIG. 2.

As shown in FIG. 2, a front end surface 33 of the endoscope front end 17 is provided with an observation window 35 of the imaging optical system, illumination openings 37A and 37B of the illumination optical system provided on both sides of the observation window 35, and a clamp opening 39. Further, an air/water sending nozzle 41 is provided thereon so as to send air or water toward the observation window 35.

In addition, as shown in FIG. 3, the endoscope front end 17 includes a front end rigid portion 43 which is made from metal such as stainless steel, an imaging portion 47 which is fixed by inserting a lens barrel 45 into a perforation hole 43a provided in the front end rigid portion 43, a metallic clamp pipe 49 which is inserted into another perforation hole 43b, a clamp tube 51 which is connected to the clamp pipe 49 and is made from a soft material, a light guide 53 of the illumination optical system, and the like.

The imaging portion 47 forms an image in such a manner that light received from the lens barrel 45 of an object lens 36 is focused on an imaging element 59 provided in the circuit board 57 through a prism 55, and outputs image information from the imaging element 59. The imaging optical system including the object lens 36, the prism 55, and the imaging element 59 is disposed in the inner space of the endoscope front end 17. In addition, the light guide 53 and the optical member such as a lens disposed in the illumination openings 37A and 37B (refer to FIG. 2) constitute the illumination optical system, and they are disposed in the inner space of the endoscope front end 17. The image information output from the imaging element 59 is transmitted to a processor (not shown) via a signal cable 58, and is processed as a display image.

The air/water sending nozzle 41 disposed toward the object lens 36 is connected to an air/water sending tube 42, and sends air or water to the object lens 36 at a desired timing. The air/water sending tube 42 is connected to the air/water sending nozzle 41 while being inserted into a perforation hole (not shown) provided in the front end rigid portion 43.

In addition, a metallic sleeve 61 is connected to the outer periphery of the front end rigid portion 43, and the metallic sleeve 61 is attached with a joint ring (not shown) disposed in the bending portion 21 (refer to FIG. 1) so that the bending portion 21 is bendable. The outer periphery of the metallic sleeve 61 is covered by a substantially tubular shell tube 63, and a front end of the front end rigid portion 43 is covered by a front end cover 65, where the shell tube 63 and the front end cover 65 are closely bonded to each other so as to prevent the occurrence of water immersion. That is, the shell tube 63 extends along the endoscope insertion unit 13 while covering the outer periphery of the front end rigid portion 43.

The shell tube 63 is made to have high thermal conductivity so that heat emission is efficiently performed by the imaging element 59, the circuit board 57 provided with the imaging element 59, or the optical member disposed from the light emission end of the light guide 53 to the illumination openings 37A and 37B. That is, since filler having high thermal conductivity is mixed in a resin, the shell tube 63 serves as a heat emission member having particularly high thermal conductivity in accordance with the endoscope insertion unit 13. In addition, the shell tube 63 has high thermal conductivity even in the thickness direction.

With this configuration, the heating of each heat emission portion is transmitted to the shell tube 63 through the front end rigid portion 43 and the metallic sleeve 61, and the heating is diffused along the endoscope insertion section 13, thereby quickly cooling the endoscope front end 17. The thermal conductivity of the shell tube 63 may be at least 1 W/(mK) in the longitudinal direction of the shell tube 63, desirably 5 to 30 W/(mK), and more desirably 10 to 30 W/(mK).

Next, a property of the shell tube 63 as the heat emission member will be described.

The shell tube 63 is a bendable soft tube which is formed as a substantially tubular shape by mixing fiber-piece-shaped filler in a resin material. The filler mixed in the resin material is a pitch-based carbon fiber, and the average diameter of the filler is 5 to 20 the length of the fiber is 20 to 500 μm, and the thermal conductivity of the fiber in the axial direction is at least 100 W/(mK). The fiber orientation direction of the filler is aligned along the longitudinal direction of the shell tube 63. For example, when the shell tube 63 is an extruded product, the fiber orientation direction is aligned along the extrusion direction of the extruding process.

A complex material obtained by mixing the material of the pitch carbon fiber and the pitch carbon fiber in the resin material is disclosed in detail in, for example, JP-A-2008-208490.

As a raw material of the carbon fiber, for example, a condensed polycyclic aromatic hydrocarbon compound such as naphthalene or phenanthrene, a condensed polycyclic hydrocarbon compound such as a coal pitch or a petroleum pitch, or the like may be exemplified. Particularly, a condensed polycyclic aromatic hydrocarbon compound such as naphthalene or phenanthrene is desirable, and a pitch exhibiting optical anisotropy, that is, a mesophase pitch is desirable. Only one type of these may be used, or two types thereof may be appropriately combined and used. However, it is desirable to only use a mesophase pitch in order to obtain a carbon fiber having high thermal conductivity.

A raw material pitch is spun by being melt-blown, and is subjected to infusibilizing and firing to thereby obtain a carbon fiber aggregate. In addition, the raw material pitch may be subjected to graphitization (crystallization) at a high temperature.

Next, a process of manufacturing and processing the shell tube 63 as the heat emission member will be described in detail.

Upon obtaining a pitch fiber from a desirable pitch raw material such as the above-described mesophase pitch by using a spinning nozzle, the fiber length or the fiber diameter of the carbon fiber may be changed. In order to change the fiber length or the fiber diameter of the carbon fiber, a ratio between the hole diameter and the land length of the nozzle hole is adjusted. In addition, the fiber length and the fiber diameter of the pitch fiber may be easily changed in accordance with a method of appropriately changing a temperature of the nozzle during spinning or a method of appropriately changing a discharge speed of a heated gas to be described later.

The pitch fiber (that is, a precursor of the carbon fiber) discharged from the nozzle hole is thinned/fiberized by discharging a gas heated in the range from 100 to 450° C. and having a linear speed of 100 to 10000 m per minute in the vicinity of the thinning point. As for the discharged gas, an inert gas such as air, nitrogen, carbon dioxide, or argon may be used. Air is desirable from the viewpoint of cost.

The pitch fiber obtained from the above-described processes has a fiber diameter (average diameter) of 5 to 20 μm. In the case where the fiber length is less than 5 μm, the shape of the fiber may not be maintained, and the productivity is poor (because most of them need to be handled as a powder). On the contrary, when the fiber length is more than 20 μm, cooling unevenness occurs due to spinning or thinning, which causes the unevenness of the fiber. Accordingly, even when the heating condition is the same, the temperature unevenness of the fiber is largely increased by infusibilizing, and there is a large concern that the fusion between fibers will partially increase.

Next, the pitch fiber obtained in this manner is subjected to infusibilizing through a known method. That is, the infusibilizing is performed by using only air, or a gas obtained by mixing air with a small amount of ozone, nitrogen dioxide, nitrogen, oxygen, iodine, or bromine. The infusibilizing temperature at that time is set to 150 to 400° C. In addition, the infusibilizing of the pitch-based fiber is desirably performed in the presence of air in consideration of safety and convenience.

Immediately after the infusibilizing, the pitch fiber is subjected to firing/graphitization at the temperature of 500 to 3500° C., and is stabilized as a graphitization (carbon) fiber, thereby obtaining the pitch-based carbon fiber. The firing of the infusibilized pitch fiber is performed under the presence of vacuum or an inert gas such as nitrogen, argon, or krypton. Generally, it is desirable to perform the firing under the presence of nitrogen at an atmospheric pressure, and cheap nitrogen is commonly used.

In addition, as a method of manufacturing a carbon fiber having a short fiber length such that the fiber length is not more than 500 μm, first, a method may be exemplified in which a pitch-based carbon fiber aggregate fired at the temperature of 500 to 1300° C. is crushed by known crushing means. The crushing method is not particularly limited, but a cutter or a crusher such as a victory mill, a jet mill, and a high-speed mill may be very appropriately used. In addition, in order to efficiently perform the crushing, a method may be desirable in which the fiber is cut into pieces in a direction perpendicular to the fiber axis by rotating a rotor provided with a blade at a high speed. The average length of the carbon fiber obtained by the crushing may be controlled by controlling the rpm of the rotor, the angle of the blade, and the like. Further, a desired fiber length can be separated by using a sifter. That is, the adjustment of the fiber diameter of the carbon fiber or the size of the fiber length may be performed by the combination of loose openings of the sifter. In addition, the separation using the sifter may be performed at an appropriate time before or after the graphitization.

The real density of the pitch-based carbon fiber is dependent on the temperature of the firing/graphitization, but is desirably in the range of 1.5 to 2.5 g/mL. More desirably, the temperature is in the range of 1.6 to 2.5 g/mL. In addition, the thermal conductivity in the fiber axial direction of the pitch-based carbon fiber is not less than 100 W/(mK), and more desirably not less than 200 W/(mK).

Next, a resin material is impregnated in the pitch-based carbon fiber, and the heat emission member is formed by a carbon fiber complex material. Here, as a resin material used for the heat emission member, one type or more of a rubber material selected from fluorine-based rubber, silicon rubber, and urethane rubber or one type or more of a fluorine-based resin selected from polytetrafluoroethylene (PTFE) and tetrafluoroethylene-perfluoroalkylvinylether copolymer (PFA) may be used.

A volume ratio of the pitch-based carbon fiber contained in a resin material as a matrix is 5 to 90 wt %, desirably 10 to 50 wt %, and more desirably 20 to 50 wt %. When the ratio of the pitch-based carbon fiber is less than 5 wt %, a desired thermal conductivity cannot be obtained. When the ratio exceeds 90 wt %, the formation thereof is difficult.

The method of impregnating the resin material in the pitch-based carbon fiber is not particular limited. However, in the case where the matrix resin is in a liquid state at a room temperature, the impregnation may be performed by using a mixing device such as a mixer. In addition, in the case where the matrix resin is in a solid state at a room temperature, the impregnation may be performed by a mixing device such as a two-axial extruder in a melted state using heating means.

In the state where the heat emission member formed by the carbon fiber complex material obtained in this manner is formed as a thin and long tubular shape, the thermal conductivity in the longitudinal direction is not less than 1 W/(mK). The method of forming the heat emission member is not particularly limited, and extruding, injecting, pressing, molding, and blowing may be exemplified. In any method, the pitch-based carbon fiber is mixed and dispersed in the matrix resin in advance, and then a mold having a desirable shape is prepared, thereby obtaining a molded product in a uniform distribution state.

In addition, a pitch-based carbon fiber aggregate is obtained by forming the pitch-based carbon fiber in a predetermined shape, the pitch-based carbon fiber is set in a mold having a desirable shape, and then a liquid or melted matrix resin is introduced thereinto. In this manner, the molded product may be formed.

Here, the thermal conductivity of the carbon fiber may be measured in accordance with a known method such as a laser flash method. In the laser flash method, specific heat capacity Cp (J/(g·K)) and thermal diffusivity α (cm²/sec) are measured. The thermal conductivity λ (W/(cmK)) is obtained from the separately measured density ρ (g/mL) by using λ=α·Cp·ρ, and is obtained by a unit conversion.

As described above, the shell tube 63 connected to the endoscope front end 17 is formed by a complex material of a high thermal conductive filler (pitch-based carbon fiber) and a resin material. The heating generated from the illumination optical system or the imaging optical system can be diffused in the longitudinal direction of the endoscope insertion unit 13 through the shell tube 63. Accordingly, it is possible to prevent an increase in the noise signal of the imaging element or deterioration in the performance by suppressing an increase in the temperature of the endoscope front end 17. In addition, even when the endoscope front end 13 is thinned, it is possible to reduce a thermal resistance in the longitudinal direction of the endoscope insertion unit 13.

In addition, since the fiber orientation direction of the filler is aligned to a direction along the longitudinal direction of the shell tube 63, it is possible to efficiently diffuse the heating of the endoscope front end 17 in the longitudinal direction of the endoscope insertion unit 13, and thus to improve the heat emission effect.

In addition, the high thermal conductive filler is not limited to the shell tube 63, but may be mixed with other members. For example, when the filler is mixed with a tube constituting member such as the clamp tube 51 and the air/water sending tube 42 shown in FIG. 3, or a coating material such as a shell of the light guide 53 enclosing a bundle of optical fibers and an insulating protection shell of the signal cable 58, it is possible to rapidly absorb the heating of the heating portion, and to diffuse the heating in the longitudinal direction of the endoscope front end 13.

Even in the case where the high thermal conductive filler is mixed with an arbitrary member, since it is not necessary to additionally provide a new heat emission member, it is possible to improve the heat emission characteristic while thinning the outer diameter of the endoscope front end 13. That is, it is not necessary to largely change the configuration of the existing endoscope. In addition, even when the complex material is formed as a thin shape so as to mix the high thermal conductive filler with the resin material, it is possible to obtain sufficient thermal conductivity.

In addition, the high thermal conductive filler may be formed as a film shape together with a resin material on the surface of the front end rigid portion 43, and may be connected to the tube constituting member or the cable coating material. Even in this case, it is possible to highly efficiently transmit the heating from the front end rigid portion 43 particularly absorbing a large amount of heat of the heat emission portion to the tube constituting member or the cable coating material.

In addition, as a material of the high thermal conductive filler, boron nitride (BN), silicon nitride (Si₃N₄), aluminum nitride (AlN), and the like may be used in addition to the pitch-based carbon fiber. In the case of using the filler made from boron nitride, since the boron nitride is comparatively compatible with a silicon resin, the silicon resin is very suitable for use as the matrix resin. In addition, in the case where the crystalline is increased, the boron nitride is formed as a scale shape, and has flexibility caused by a loose layered structure in which the scales are overlapped with each other. For this reason, in the complex material containing the boron nitride, contact between particles is satisfactory, and the thermal conductivity of the complex material is more improved than other filler materials. In addition, the boron nitride has a characteristic in which the thermal conductivity of a surface direction of the scale is remarkably increased compared to that in the thickness direction. When the orientation direction of the scales is aligned in accordance with the characteristic, it is possible to improve the thermal conductivity in a direction along the endoscope insertion unit.

In the case of using the silicon nitride, since the silicon nitride is excellent from the viewpoint of economy, purity, low-temperature expansion rate, and chemical safety, and is easily handled in the industrial field, it is possible to stably use the complex material at a low cost.

In the case of using the aluminum nitride, since the aluminum nitride has particularly high thermal conductivity, and the thermal conductivity of the complex material is improved when the charge amount of the complex material is increased, the diffusion of the heating is highly efficiently performed.

Here, a complex material is formed by the mixture of silicon-based rubber and a high thermal conductive filler having thermal conductivity of 600 W/mk, a fiber length of 30 to 80 μm, and a diameter of 5 to 10 μm. The thermal conductivity characteristic is observed and analyzed by a rubber heater and a thermo viewer. That is, on the basis of the observed temperature distribution, the thermal conductivity is calculated backward through a thermal resistance circuit network or a three-dimensional thermal fluid analysis simulation.
Mixture Ratio: about 50 wt %
Measurement Result of Thermal Conductivity: about 10 W/mk

As the high thermal conductive filler, for example, “Raheama (trademark)” produced by Teijin Corporation may be very appropriately exemplified. As the application purpose of the “Raheama”, there is a heat emission (thermal conductive) sheet formed by a matrix material of rubber/elastomer. In this case, the thermal conductivity is not less than 20 W/mk.

In addition, on the basis of the analysis result of the thermal conductivity characteristic and the example of the application purpose of the “Raheama”, even in the case where the matrix is formed by urethane rubber or fluorine rubber, thermal conductivity of 10 to 20 W/mk may be obtained.

According to the thermal conductivity characteristic of the matrix material, in the case where the matrix material is used as the shell tube 63 (refer to FIG. 3), it is possible to reduce an increase in the temperature of the endoscope front end by 10 to 20%. In addition, even in the case where the matrix material is used as the shells of the air/water sending tube 42, the clamp tube 51, and the light guide 53 and the insulating protection shell of the signal cable 58, it is possible to obtain a temperature increase reduction effect of about 5%.

As described above, the following contents are disclosed in the specification.

(1) An endoscope includes an endoscope insertion unit which has a front end rigid portion constituting a front end of the endoscope insertion unit. The front end rigid portion is provided with an illumination optical system irradiating an observation target area with illumination light and an imaging optical system having an imaging element imaging the observation target area. The endoscope also includes a heat emission member which is connected to the front end rigid portion and extends in the longitudinal direction of the endoscope insertion unit. The heat emission member is formed by mixing fiber-piece-shaped fillers with a resin material.

According to the endoscope, it is possible to diffuse the heating generated from the illumination optical system or the imaging optical system in the longitudinal direction of the endoscope insertion unit through the heat emission member. Accordingly, it is possible to prevent an increase in the noise signal of the imaging element or deterioration in the performance by suppressing an increase in the temperature of the endoscope front end. In addition, even when the endoscope front end unit is further thinned, it is possible to sufficiently reduce a thermal resistance in the longitudinal direction of the endoscope insertion unit.

(2) The endoscope according to (1), the heat emission member includes a substantially tubular shell tube which covers an outer periphery of the front end rigid portion and extends along the endoscope insertion unit.

According to the endoscope, it is possible to highly efficiently diffuse the heating generated from the illumination optical system or the imaging optical system in the longitudinal direction of the endoscope insertion unit in such a manner that the shell tube covering the outer periphery of the front end rigid portion absorbs the heating. When the shell tube is used as the shell of the endoscope insertion unit, the heat exchange property is increased due to contact or the like with the outside of the endoscope, and hence the heat emission property is improved.

(3) The endoscope according to (1) or (2), wherein the heat emission member includes at least one tube constituting member which is disposed inside the endoscope insertion unit.

According to the endoscope, it is possible to highly efficiently diffuse the heating generated from the illumination optical system or the imaging optical system in the longitudinal direction of the endoscope insertion unit in such a manner that the heating is absorbed by the tube constituting member connected to the front end rigid portion.

(4) The endoscope according to any one of (1) to (3), wherein the heat emission member includes at least one cable coating material which is disposed inside the endoscope insertion unit.

According to the endoscope, it is possible to highly efficiently diffuse the heating generated from the illumination optical system or the imaging optical system in the longitudinal direction of the endoscope insertion unit in such a manner that the heating is absorbed by the cable coating material.

(5) The endoscope according to any one of (1) to (4), wherein each of the fillers is a fiber piece which has an average diameter of 5 to 20 μm, a fiber length of 20 to 500 μm, and a thermal conductivity of at least 100 W/(mK) in a fiber axial direction.

According to the endoscope, it is possible to improve the thermal diffusion property in such a manner that the fiber piece having a predetermined size and high thermal conductivity is mixed in a resin material.

(6) The endoscope according to (5), wherein the filler is formed by a pitch-based carbon fiber.

According to the endoscope, it is possible to form the heat emission member having an excellent machine characteristic or thermal conductivity by using the pitch-based carbon fiber.

(7) The endoscope according to any one of (1) to (6), wherein the resin material is formed by a rubber material.

According to the endoscope, it is possible to improve the flexibility of the heat emission member by using the resin material as the rubber material, and thus to improve the shock resistance.

(8) The endoscope according to (7), wherein the rubber material is at least one type of material selected from fluorine-based rubber, silicon rubber, and urethane rubber.

According to the endoscope, it is possible to form the heat emission member having excellent thermal resistance by using the fluorine-based rubber or silicon rubber. Also, it is possible to form the heat emission member having excellent abrasion resistance by using the urethane rubber.

(9) The endoscope according to any one of (1) to (6), wherein the resin material is a fluorine-based resin.

According to the endoscope, it is possible to improve the thermal resistance of the heat emission member by using the fluorine-based resin.

(10) The endoscope according to (9), wherein the fluorine-based resin is at least one type of a material selected from polytetrafluoroethylene (PTFE) and tetrafluoroethylene-perfluoroalkylvinylether copolymer (PFA).

According to the endoscope, it is possible to form the heat emission member having excellent thermal resistance and abrasion resistance by using PTFE and PFA.

(11) The endoscope according to any one of (1) to (10), wherein at least a part of the fillers inside the resin material are dispersed so as to contact each other, and thermal conductivity in the longitudinal direction of the heat emission member is at least 1 W/(mK).

According to the endoscope, it is possible to improve the thermal conductivity by the contact between the fillers inside the resin material. Also, it is possible to sufficiently absorb the heating generated from the illumination optical system or the imaging optical system by using the heat emission member.

(12) The endoscope according to any one of (1) to (11), wherein a fiber orientation direction of the filler inside the heat emission member is a direction along the longitudinal direction of the heat emission member.

According to the endoscope, since the thermal conductivity in the longitudinal direction of the heat emission member is improved, it is possible to improve the thermal diffusion property of the endoscope front end.

(13) The endoscope according to (12), wherein the heat emission member is formed by an extruded product in which the fiber orientation direction of the filler is aligned to the extrusion direction.

According to the endoscope, the fiber orientation direction of the filler is easily aligned to the extrusion direction by extruding the heat emission member.

What is claimed is:

1. An endoscope comprising:
an endoscope insertion unit that includes a front end rigid portion constituting a front end of the endoscope insertion unit, the front end rigid portion being provided with (i) an illumination optical system irradiating an observation target area with illumination light and (ii) an imaging optical system having an imaging element imaging the observation target area; and
a heat emission member that is connected to the front end rigid portion, and that extends in the longitudinal direction of the endoscope insertion unit,
wherein the heat emission member is formed by mixing fiber-piece-shaped fillers with a resin material.

2. The endoscope according to claim 1,
wherein the heat emission member includes a substantially tubular shell tube which covers an outer periphery of the front end rigid portion, and extends along the endoscope insertion unit.

3. The endoscope according to claim 1,
wherein the heat emission member includes at least one tube constituting member which is disposed inside the endoscope insertion unit.

4. The endoscope according to claim 1,
wherein the heat emission member includes at least one cable coating material which is disposed inside the endoscope insertion unit.

5. The endoscope according to claim 1,
wherein each of the fillers is a fiber piece which has an average diameter of 5 to 20 μm, a fiber length of 20 to 500 μm, and a thermal conductivity of at least 100 W/(mK) in a fiber axial direction.

6. The endoscope according to claim 5,
wherein the filler is formed by a pitch-based carbon fiber.

7. The endoscope according to claim 1,
wherein the resin material is formed by a rubber material.

8. The endoscope according to claim 7,
wherein the rubber material is at least one type of material selected from fluorine-based rubber, silicon rubber, and urethane rubber.

9. The endoscope according to claim 1,
wherein the resin material is a fluorine-based resin.

10. The endoscope according to claim 9,
wherein the fluorine-based resin is at least one type of a material selected from polytetrafluoroethylene (PTFE) and tetrafluoroethylene-perfluoroalkylvinylether copolymer (PFA).

11. The endoscope according to claim 1,
wherein at least a part of the fillers inside the resin material are dispersed so as to contact each other, and thermal conductivity in the longitudinal direction of the heat emission member is at least 1 W/(mK).

12. The endoscope according to claim 1,
wherein a fiber orientation direction of the filler inside the heat emission member is a direction along the longitudinal direction of the heat emission member.

13. The endoscope according to claim 12,
wherein the heat emission member is formed by an extruded product in which the fiber orientation direction of the filler is aligned to the extrusion direction.
